SEQUENCE LISTING (14 sequences)

SEQ ID NO: 1 - DNA, 90 bp, Homo sapiens
ccgggcccctgtgagcatcttaccggacagtgctggatttcccagcttgactctaacactgtctggtaacgatgttcaaaggtgacccgc

SEQ ID NO: 2 - DNA, 98 bp, Homo sapiens
acccaaaccctaggtctgctgactcctagtccagggctcgtgatggctggtgggccctgaacgaggggtctggaggcctgggtttgaatatcgacagc

SEQ ID NO: 3 - DNA, 69 bp, Homo sapiens
gggatactcaaaatgggggcgctttcctttttgtctgtactgggaagtgcttcgattttggggtgtccc

SEQ ID NO: 4 - DNA, 97 bp, Homo sapiens
aacttaacatcatgctacctctttgtatcatattttgttattctggtcacagaatgacctagtattctgtaccagggaaggtagttcttaactatat

SEQ ID NO: 5 - DNA, 93 bp, Homo sapiens
ccttccggcgtcccaggcggggcgccgcgggaccgccctcgtgtctgtggcggtgggatcccgcggccgtgttttcctggtggcccggccatg

SEQ ID NO: 6 - DNA, 114 bp, Homo sapiens
tttaggcgctgatgaaagtggagttcagtagacagcccttttcaagccctacgagaaactggggtttctggaggagaaggaaggtgatgaaggatctgttctcgtgagcctgaa

SEQ ID NO: 7 - DNA, 85 bp, Homo sapiens
agggtagagggatgagggggaaagttctatagtcctgtaattagatctcaggactatagaactttccccctcatccctctgccct

SEQ ID NO: 8 - DNA, 80 bp, Homo sapiens
ctgggggctccaaagtgctgttcgtgcaggtagtgtgattacccaacctactgctgagctagcacttcccgagcccccgg

SEQ ID NO: 9 - DNA, 82 bp, Homo sapiens
cctgccggggctaaagtgctgacagtgcagatagtggtcctctccgtgctaccgcactgtgggtacttgctgctccagcagg

SEQ ID NO: 10 - DNA, 65 bp, Homo sapiens
ctgttaatgctaatcgtgataggggtttttgcctccaactgactcctacatattagcattaacag

SEQ ID NO: 11 - DNA, 82 bp, Homo sapiens
ggcctgcccgacactctttccctgttgcactactataggccgctgggaagcagtgcaatgatgaaagggcatcggtcaggtc

SEQ ID NO: 12 - DNA, 71 bp, Homo sapiens
gcgactgtaaacatcctcgactggaagctgtgaagccacagatgggctttcagtcggatgtttgcagctgc

SEQ ID NO: 13 - DNA, 86 bp, Homo sapiens
actgctaacgaatgctctgactttattgcactactgtactttacagctagcagtgcaatagtattgtcaaagcatctgaaagcagg

SEQ ID NO: 14 - DNA, 98 bp, Homo sapiens
ttgaggccttaaagtactgtagcagcacatcatggtttacatgctacagtcaagatgcgaatcattatttgctgctctagaaatttaaggaaattcat

(19) United States
(12) Patent Application Publication
(10) Pub. No.: US 2014/0031258 A1
(43) Pub. Date: Jan. 30, 2014

(54) SERUM-BASED MIRNA MICROARRAY AND ITS USE IN DIAGNOSIS AND TREATMENT OF BARRETT'S ESOPHAGUS (BE) AND ESOPHAGEAL ADENOCARCINOMA (EAC)

(71) Applicants: Stephen J. Meltzer, Lutherville, MD (US); Yulan Cheng, Ellicott City, MD (US); Jee-Hoon Song, Baltimore, MD (US)

(72) Inventors: Stephen J. Meltzer, Lutherville, MD (US); Yulan Cheng, Ellicott City, MD (US); Jee-Hoon Song, Baltimore, MD (US)

(73) Assignee: THE JOHNS HOPKINS UNIVERSITY, Baltimore, MD (US)

(21) Appl. No.: 14/008,057

(22) PCT Filed: Mar. 26, 2012
(86) PCT No.: PCT/US2012/030518

§ 371 Date: Oct. 21, 2013

Related U.S. Application Data
(60) Provisional application No. 61/468,194, filed on Mar. 28, 2011.

(51) Int. Cl. C12Q 1/68

(52) U.S. Cl. CPC C12Q 1/6886
USPC 506/9; 506/16

(57) ABSTRACT

Robust and reliable molecular diagnostic screening tools for early detection of esophageal and gastrointestinal tract cancers and pre-cancerous lesions, such as Barrett's Esophagus, and esophageal adenocarcinoma are provided. Included in the invention is an array of miRNA probes specific for identifying, diagnosing and prognosticating esophageal and gastrointestinal tract cancers and pre-cancerous lesions in subjects from blood or serum samples. A biochip comprising the array as well as methods for its use are also provided.

### REFERENCE TO RELATED APPLICATIONS

This application claims the benefit of U.S. Provisional Patent Application No. 61/468,194, filed on Mar. 28, 2011, which is hereby incorporated by reference for all purposes as if fully set forth herein.

### STATEMENT OF GOVERNMENTAL INTEREST

This invention was made with U.S. government support under grant no. CAI46799-01AI. The U.S. government has certain rights in the invention.

### BACKGROUND OF THE INVENTION

As in the case of most diseases, in order to improve the prognosis of patients, a diagnosis at an early stage is crucial. For example, esophageal cancer (EC), the 8th-most common malignancy and 6^{th} most frequent cause of cancer death worldwide, exhibits highly aggressive behavior. Barrett's esophagus (BE) is the obligate precursor lesion of esophageal adenocarcinoma (EAC), one of the two major histologic subtypes of EC. Early detection and close periodic surveillance of BE is the best means to intervene in BE-associated neoplastic progression (BN). Existing methods for detecting EC are endoscopic biopsy and histopathological examinations, but they are limited due to their invasive nature and inability to be applied in large-scale studies.

As many as 3 million Americans harbor BE; however, 40% or more of EACs are diagnosed in subjects lacking any previous symptoms, and only 5% of patients presenting with EAC carry an antecedent diagnosis of BE. Early detection and close periodic surveillance of BE is the best means to intervene in BE-associated neoplastic progression (BN). Nevertheless, EAC develops in only 0.5%-1.0% of previously diagnosed BE patients annually. Thus, most patients presenting with EAC have not benefited from endoscopic (EGO) surveillance of BE. EGO is unsuitable and impractical for population-based screening or detection of asymptomatic BN. Furthermore, performing EGO based only on symptoms risks missing patients with asymptomatic BE and/or EAC. Noninvasive diagnosis of BE would enroll a higher proportion of individuals with BE into EGO surveillance programs before they develop EAC, increasing BN diagnosis at earlier, more survivable stages. At the same time, noninvasive diagnosis of EAC would also improve outcome.

MicroRNAs (miRNAs or miRs) are short RNA oligonucleotides of approximately 22 nucleotides that are involved in gene regulation. MicroRNAs regulate gene expression by targeting mRNAs for cleavage or translational repression. Although miRNAs are present in a wide range of species including C. elegans, Drosophila and humans, they have only recently been identified. More importantly, the role of miRNAs in the development and progression of disease has only recently become appreciated. Deregulated miRNA expression is implicated in onset and progression of different diseases including, but not limited to embryonic malformations and cancers.

As a result of their small size, miRNAs have been difficult to identify using standard methodologies. A limited number of miRNAs have been identified by extracting large quantities of RNA. MiRNAs have also been identified that contribute to the presentation of visibly discernable phenotypes. Expression array data shows that miRNAs are expressed in different developmental stages or in different tissues. The restriction of miRNAs to certain tissues or at limited developmental stages indicates that the miRNAs identified to date are likely only a small fraction of the total miRNAs.

Therefore, there still exists an imperative need to develop robust and reliable molecular diagnostic screening tools for the early detection of BE and/or EAC that will enhance the likelihood of cure and reduce the incremental costs for the treatment of advanced disease.

### SUMMARY OF THE INVENTION

In one or more embodiments, the present invention provides an array of miRNA biomarkers that are detectable in the blood or serum of subjects, which comprise a noninvasive diagnostic technology that is sufficiently sensitive to detect oncogenic, cancerous, premalignant or metaplastic changes in the gastrointestinal tract of a mammalian subject.

In accordance with an embodiment, the present invention provides an array of oligonucleotide probes for identifying miRNAs, or portions or fragments thereof, in a sample, comprising probes that each selectively bind a mature miRNA, or a portion or fragment thereof, and a platform, wherein the probes are immobilized on the platform, wherein at least two probes selectively bind a human miRNA selected from human miRNAs comprising sequences of SEQ ID NOS: 1-7 and 10-14, or portions or fragments thereof, or at least two probes are selected from probes comprising sequences of SEQ ID NOS: 1-7 and 10-14, or portions or fragments thereof.

In accordance with another embodiment, the present invention provides a biochip comprising a solid substrate, and further comprising at least two oligonucleotide probes which selectively bind a human miRNA selected from human miRNAs comprising sequences of SEQ ID NOS: 1-14, or portions or fragments thereof, or at least two probes are selected from probes comprising sequences of SEQ ID NOS: 1-14, or portions or fragments thereof, which are capable of hybridizing to a target sequence under stringent hybridization conditions and attached at spatially defined address on the substrate.

In accordance with an further embodiment, the present invention provides a method of determining oncogenic, cancerous, premalignant or metaplastic changes the esophagus or gastrointestinal tract of a mammalian subject comprising (a) extracting miRNA from a sample obtained from a mammalian subject, (b) contacting the miRNA from (a) with the array or the biochip as described above, (c) performing an analysis using the array or biochip of b) to determine expression of at least one miRNA obtained from the sample, and (d) comparing the expression of at least two or more miRNA obtained from the sample tissue with the expression of at least one miRNA obtained from a control sample, wherein a detectable change in the expression of at least two or more miRNA obtained from the sample compared to control is indicative of oncogenic, cancerous, premalignant or metaplastic changes in the gastrointestinal tract of a mammalian subject.

In accordance with still another embodiment, the present invention provides a method of staging the oncogenic, cancerous, premalignant or metaplastic changes in the esophagus or gastrointestinal tract of a mammalian subject comprising a) obtaining a sample from the subject, b) contacting the RNA from (a) with the array or biochip described above, c) determining the amount of at least two miRNA selected from the group consisting of hsa-miR-200a (SEQ ID NO: 1), hsa-miR-345 (SEQ ID NO: 2), hsa-miR-373 (SEQ ID NO: 3), hsa-miR-630 (SEQ ID NO: 4), hsa-miR-663 (SEQ ID NO: 5), hsa-miR-765 (SEQ ID NO: 6), hsa-miR-625 (SEQ ID NO: 7), hsa-miR-93 (SEQ ID NO: 8), hsa-miR-106b (SEQ ID NO: 9), hsa-miR-155 (SEQ ID NO: 10), hsa-miR-130b (SEQ ID NO: 11), hsa-miR-30a (SEQ ID NO: 12), hsa-miR-301a (SEQ ID NO: 13), hsa-miR-15b (SEQ ID NO: 14), or portions or fragments thereof, or the amount of a precursor molecule of the at least one miRNA, or portions or fragments thereof, in the sample from the subject, d) comparing the amount of the at least two miRNA or the amount of a precursor molecule of the at least two miRNA of a) with at least one or more reference or control amounts, and wherein when a detectable change in the amount of at least two miRNA or portions or fragments thereof, obtained from the sample compared to the reference or control, the stage of the oncogenic, cancerous, premalignant or metaplastic changes in the gastrointestinal tract of a mammalian subject is determined.

In an embodiment, the present invention provides a method of determining oncogenic, cancerous, premalignant or metaplastic changes the esophagus or gastrointestinal tract of a mammalian subject comprising, (a) extracting miRNA from a sample obtained from a mammalian subject, (b) determining the expression of at least two miRNA obtained from the sample, and (c) comparing the expression of at least two miRNA obtained from the sample tissue with the expression of at least one miRNA obtained from a control sample, wherein a detectable change in the expression of at least one miRNA obtained from the sample compared to control is indicative of oncogenic, cancerous, premalignant or metaplastic changes in the gastrointestinal tract of a mammalian subject.

In accordance with another embodiment, the present invention provides a method of staging the oncogenic, cancerous, premalignant or metaplastic changes in the esophagus or gastrointestinal tract of a mammalian subject comprising a) obtaining a sample from the subject, b) determining the amount of at least two miRNA selected from the group consisting of hsa-miR-200a, hsa-miR-345, hsa-miR-373, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301a, hsa-miR-15b, or portions or fragments of any of these miRNAs thereof, or the amount of a precursor molecule of the at least two miRNA in the sample from the subject, c) comparing the amount of the at least two miRNA or the amount of a precursor molecule of the at least two miRNA of a) with at least one or more reference or control amounts, and wherein when a detectable change in the amount of at least two miRNA obtained from the sample compared to the reference or control, the stage of the oncogenic, cancerous, premalignant or metaplastic changes in the gastrointestinal tract of a mammalian subject is determined.

In accordance with a further embodiment, the present invention provides a method for diagnosing the progression the oncogenic, cancerous, premalignant or metaplastic changes in the esophagus or gastrointestinal tract of a mammalian subject comprising a) obtaining a sample from the subject, b) determining the amount of at least two miRNA selected from the group consisting of hsa-miR-200a, hsa-miR-345, hsa-miR-373, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301a, hsa-miR-15b or portions or fragments of any of these miRNAs thereof, or the amount of a precursor molecule of the at least two miRNA in the sample from the subject, c) comparing the amount of the at least two miRNA or the amount of a precursor molecule of the at least two miRNA of a) with at least one or more reference or control amounts, and wherein when a detectable change in the amount of at least two miRNA obtained from the sample compared to the reference or control, the progression of the oncogenic, cancerous, premalignant or metaplastic changes in the gastrointestinal tract of a mammalian subject is determined.

In accordance with yet another embodiment, the present invention provides a use of at least two miRNA selected from the group consisting hsa-miR-200a, hsa-miR-345, hsa-miR-373, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301a, hsa-miR-15b or portions or fragments of any of these miRNAs thereof, or of a precursor molecule thereof in a sample from a subject suffering from oncogenic, cancerous, premalignant or metaplastic changes in the gastrointestinal tract for identifying a subject being susceptible to gastrointestinal cancer therapy.

### BRIEF DESCRIPTION OF THE DRAWINGS

FIG. 1 is a table of miR-array data generated from human samples that was normalized by the array control small RNA called Hurs.

FIG. 2 is a table of miR-array data generated from human samples that was normalized by Agilent's GeneSpring GX 11.5 software.

FIG. 3 is a table of miR-array data generated from cell line samples that was normalized by the array control small RNA called Hurs.

### DETAILED DESCRIPTION OF THE INVENTION

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In an embodiment, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids used as primers in embodiments of the present invention can be constructed based on chemical synthesis and/or enzymatic ligation reactions using procedures known in the art. See, for example, Sambrook et al. (eds.), Molecular Cloning, A Laboratory Manual, 3^{rd} Edition, Cold Spring Harbor Laboratory Press, New York (2001) and Ausubel et al., Current Protocols in Molecular Biology, Greene Publishing Associates and John Wiley & Sons, NY (1994). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleotide sequences used herein are those which hybridize under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C.

The term “isolated and purified” as used herein means a protein that is essentially free of association with other proteins or polypeptides, e.g., as a naturally occurring protein that has been separated from cellular and other contaminants by the use of antibodies or other methods or as a purification product of a recombinant host cell culture.

The term “biologically active” as used herein means an enzyme or protein having structural, regulatory, or biochemical functions of a naturally occurring molecule.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In accordance with one or more embodiments of the present invention, it will be understood that the types of cancer diagnosis which may be made, using the methods provided herein, is not necessarily limited. For purposes herein, the cancer can be any cancer. As used herein, the term “cancer” is meant any malignant growth or tumor caused by abnormal and uncontrolled cell division that may spread to other parts of the body through the lymphatic system or the blood stream.

The cancer can be a metastatic cancer or a non-metastatic (e.g., localized) cancer. As used herein, the term “metastatic cancer” refers to a cancer in which cells of the cancer have metastasized, e.g., the cancer is characterized by metastasis of a cancer cells. The metastasis can be regional metastasis or distant metastasis, as described herein.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of diagnosis, staging, screening, or other patient management, including treatment or prevention of cancer in a mammal Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

“Complement” or “complementary” as used herein to refer to a nucleic acid may mean Watson-Crick (e.g., A-T/U and C-G) or Hoogsteen base pairing between nucleotides or nucleotide analogs of nucleic acid molecules.

“Differential expression” may mean qualitative or quantitative differences in the temporal and/or cellular gene expression patterns within and among cells and tissue. Thus, a differentially expressed gene may qualitatively have its expression altered, including an activation or inactivation, in, e.g., normal versus disease tissue. Genes may be turned on or turned off in a particular state, relative to another state thus permitting comparison of two or more states. A qualitatively regulated gene may exhibit an expression pattern within a state or cell type which may be detectable by standard techniques. Some genes may be expressed in one state or cell type, but not in both. Alternatively, the difference in expression may be quantitative, e.g., in that expression is modulated, either up-regulated, resulting in an increased amount of transcript, or down-regulated, resulting in a decreased amount of transcript. The degree to which expression differs need only be large enough to quantify via standard characterization techniques such as expression arrays, quantitative reverse transcriptase PCR, northern analysis, and RNase protection.

“Identical” or “identity” as used herein in the context of two or more nucleic acids or polypeptide sequences may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

“Substantially complementary” used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

“Target” as used herein can mean an oligonucleotide or portions or fragments thereof, which may be bound by one or more probes under stringent hybridization conditions. “Target” as used herein may also mean a specific miRNA or portions or fragments thereof, which may be bound by one or more probes under stringent hybridization conditions.

In accordance with an embodiment, the present invention provides an array of oligonucleotide probes for identifying miRNAs in a sample, comprising probes that each selectively bind a mature miRNA, and a platform, wherein the probes are immobilized on the platform, wherein at least two probes selectively bind a human miRNA selected from human miRNAs consisting of sequences of SEQ ID NOS: 1-7 and 10-14 or portions or fragments thereof, or at least two probes are selected from probes consisting of sequences of SEQ ID NOS: 1-7 and 10-14, for example, hsa-miR-200a (SEQ ID NO: 1), hsa-miR-345 (SEQ ID NO: 2), hsa-miR-373 (SEQ ID NO: 3), hsa-miR-630 (SEQ ID NO: 4), hsa-miR-663 (SEQ ID NO: 5), hsa-miR-765 (SEQ ID NO: 6), hsa-miR-625 (SEQ ID NO: 7), hsa-miR-155 (SEQ ID NO: 10), hsa-miR-130b (SEQ ID NO: 11), hsa-miR-30a (SEQ ID NO: 12), hsa-miR-301a (SEQ ID NO: 13), hsa-miR-15b (SEQ ID NO: 14) or portions or fragments of any of these miRNAs thereof.

In another embodiment, the present invention provides an array of oligonucleotide probes for identifying miRNAs in a sample, comprising probes that each selectively bind a mature miRNA, and a platform, wherein the probes are immobilized on the platform, wherein at least three probes selectively bind a human miRNAs consisting of sequences of SEQ ID NOS: 1-7 and 10-14; or at least three probes are selected from probes consisting of sequences of SEQ ID NOS: 1-7 and 10-14. It will be understood by those of ordinary skill that the array can bind any number of oligonucleotide probes, including 4, 5, 6, 7, 8, 9, 10, 11, 12, 13, and 14 probes at one time.

The nucleic acids of the present invention may also comprise a sequence of a miRNA or a variant thereof. The miRNA sequence may comprise from 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90 and up to 100 nucleotides. The sequence of the miRNA may be the first 13-33 nucleotides of the pre-miRNA. The sequence of the miRNA may also be the last 13-33 nucleotides of the pre-miRNA. The sequence of the miRNA may comprise the sequence of SEQ ID NOS: 1-14 or portions or fragments thereof.

A probe is also provided comprising a nucleic acid described herein. Probes may be used for screening and diagnostic methods, as outlined below. The probes may be attached or immobilized to a solid substrate or apparatus, such as a biochip.

The probe may have a length of from 8 to 500, 10 to 100 or 20 to 60 nucleotides. The probe may also have a length of at least 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 260, 280 or 300 nucleotides. The probe may further comprise a linker sequence of from 10-60 nucleotides.

A biochip is also provided. The biochip is an apparatus which, in certain embodiments, comprises a solid substrate comprising an attached probe or plurality of probes described herein. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. In an embodiment, two or more probes per target sequence are used. The probes may be capable of hybridizing to target sequences associated with a single disorder.

The probes may be attached to the biochip in a wide variety of ways, as will be appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as a flexible foam, including closed cell foams made of particular plastics.

The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linkers. The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

A method of identifying a nucleic acid associated with a disease or a pathological condition is also provided. The method comprises measuring a level of the nucleic acid in a sample that is different than the level of a control. In accordance with an embodiment, the nucleic acid is a miRNA and the detection may be performed by contacting the sample with a probe or biochip described herein and detecting the amount of hybridization. PCR may be used to amplify nucleic acids in the sample, which may provide higher sensitivity.

The level of the nucleic acid in the sample may also be compared to a control cell (e.g., a normal cell) to determine whether the nucleic acid is differentially expressed (e.g., overexpressed or underexpressed). The ability to identify miRNAs that are differentially expressed in pathological cells compared to a control can provide high-resolution, high-sensitivity datasets which may be used in the areas of diagnostics, prognostics, therapeutics, drug development, pharmacogenetics, biosensor development, and other related areas.

The expression level of a disease-associated nucleic acid or miRNA provides information in a number of ways. For example, a differential expression of a disease-associated nucleic acid compared to a control may be used as a diagnostic that a patient suffers from the disease. Expression levels of a disease-associated nucleic acid may also be used to monitor the treatment and disease state of a patient. Furthermore, expression levels of a disease-associated miRNA may allow the screening of drug candidates for altering a particular expression profile or suppressing an expression profile associated with disease.

A target nucleic acid or portions or fragments thereof, may be detected and levels of the target nucleic acid measured by contacting a sample comprising the target nucleic acid with a biochip comprising an attached probe sufficiently complementary to the target nucleic acid and detecting hybridization to the probe above control levels.

The target nucleic acid or portions or fragments thereof, may also be detected by immobilizing the nucleic acid to be examined on a solid support such as nylon membranes and hybridizing a labeled probe with the sample. Similarly, the target nucleic or portions or fragments thereof, may also be detected by immobilizing the labeled probe to a solid support and hybridizing a sample comprising a labeled target nucleic acid. Following washing to remove the non-specific hybridization, the label may be detected.

The target nucleic acid or portions or fragments thereof, may also be detected in situ by contacting permeabilized cells or tissue samples with a labeled probe to allow hybridization with the target nucleic acid. Following washing to remove the non-specifically bound probe, the label may be detected.

The detection of the target nucleic acid, or portions or fragments thereof, can be through direct hybridization assays or can comprise sandwich assays, which include the use of multiple probes, as is generally known in the art.

A variety of hybridization conditions may be used, including high, moderate and low stringency conditions as outlined above. The assays may be performed under stringency conditions which allow hybridization of the probe only to the target. Stringency can be controlled by altering a step parameter that is a thermodynamic variable, including, but not limited to, temperature, formamide concentration, salt concentration, chaotropic salt concentration pH, or organic solvent concentration.

Hybridization reactions may be accomplished in a variety of ways. Components of the reaction may be added simultaneously, or sequentially, in different orders. In addition, the reaction may include a variety of other reagents. These include salts, buffers, neutral proteins, e.g., albumin, detergents, etc. which may be used to facilitate optimal hybridization and detection, and/or reduce non-specific or background interactions. Reagents that otherwise improve the efficiency of the assay, such as protease inhibitors, nuclease inhibitors and anti-microbial agents may also be used as appropriate, depending on the sample preparation methods and purity of the target.

A kit is also provided comprising an array of oligonucleotides as described herein, or portions or fragments thereof, as well as a biochip as described herein, along with any or all of the following: assay reagents, buffers, probes and/or primers, and sterile saline or another pharmaceutically acceptable emulsion and suspension base. In addition, the kits may include instructional materials containing directions (e.g., protocols) for the practice of the methods described herein.

In accordance with another embodiment of the present invention, it will be understood that the term “biological sample” or “biological fluid” includes, but is not limited to, any quantity of a substance from a living or formerly living patient or mammal Such substances include, but are not limited to, blood, serum, plasma, urine, cells, organs, tissues, bone, bone marrow, lymph, lymph nodes, synovial tissue, chondrocytes, synovial macrophages, endothelial cells, and skin. In a preferred embodiment, the fluid is blood or serum.

A method of diagnosis is also provided. The method comprises detecting a differential expression level of two or more disease-associated miRNAs in a biological sample. The sample may be derived from a subject. Diagnosis of a disease state in a subject may allow for prognosis and selection of therapeutic strategy. Further, the developmental stage of cells may be classified by determining temporarily expressed disease-associated miRNAs.

In situ hybridization of labeled probes to tissue arrays may be performed. When comparing the levels of miRNA expression between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or may be predictive of outcomes.

In accordance with an embodiment, the present invention provides an array of oligonucleotide probes for identifying miRNAs in a sample, comprising: probes that each selectively bind a mature miRNA; and a platform, wherein the probes are immobilized on the platform; wherein at least one probe selectively binds a human miRNA selected from human miRNAs comprising sequences of SEQ ID NOS: 1-14 or a portion or fragment thereof; or at least one probe is selected from probes comprising sequences of SEQ ID NOS: 1-14 or a portion or fragment thereof.

Exemplary biochips of the present invention include an organized assortment of oligonucleotide probes described above immobilized onto an appropriate platform. Each probe selectively binds a miRNA in a sample. In certain embodiments, each probe of the biochip selectively binds a biologically active mature miRNA in a sample.

In accordance with another embodiment, the biochip of the present invention can also include one or more positive or negative controls. For example, oligonucleotides with randomized sequences can be used as positive controls, indicating orientation of the biochip based on where they are placed on the biochip, and providing controls for the detection time of the biochip when it is used for detecting miRNAs in a sample.

Embodiments of the biochip can be made in the following manner. The oligonucleotide probes to be included in the biochip are selected and obtained. The probes can be selected, for example, based on a particular subset of miRNAs of interest. The probes can be synthesized using methods and materials known to those skilled in the art, or they can be synthesized by and obtained from a commercial source, such as GeneScript USA (Piscataway, N.J.).

Each discrete probe is then attached to an appropriate platform in a discrete location, to provide an organized array of probes. Appropriate platforms include membranes and glass slides. Appropriate membranes include, for example, nylon membranes and nitrocellulose membranes. The probes are attached to the platform using methods and materials known to those skilled in the art. Briefly, the probes can be attached to the platform by synthesizing the probes directly on the platform, or probe-spotting using a contact or non-contact printing system. Probe-spotting can be accomplished using any of several commercially available systems, such as the GeneMachines™ OmniGrid (San Carlos, Calif.).

The miRNA sample can be amplified and labeled as is appropriate or desired. If amplification is desired, methods known to those skilled in the art can be applied. The miRNA samples can be labeled using various methods known to those skilled in the art. In accordance with an embodiment, the miRNA samples are labeled with digoxigenin using a Digoxigenin (DIG) Nucleotide Tailing Kit (Roche Diagnostics Corporation, Indianapolis, Ind.) in a GeneAmp® PCR System 9700 (Applied Biosystems, Foster City, Calif.).

The labeled miRNA sample is incubated with the biochip, allowing the miRNAs in the sample to hybridize with a probe specific for the miRNAs in the sample. In certain embodiments, the labeled miRNA sample is added to a DIG Easy Hyb Solution or Hybrid Easy Buffer (Roche Diagnostics Corporation, Indianapolis, Ind.) that has been preheated to hybridization temperature. The miRNA sample is the incubated with the biochip in the solution, for example, for about 4 hours to about 24 hours.

The miRNAs in the sample can be detected, identified, and quantified in the following manner. After the miRNA sample has been incubated with the biochip for an appropriate time period, the biochip is washed with a series of washing buffers, and then incubated with a blocking buffer. When Digoxigenin (DIG) labeling of the miRNA samples has been used, the biochip is then incubated with an Anti-DIG-AP antibody (Roche Diagnostics Corporation, Indianapolis, Ind.). The biochip is them washed with washing buffer and incubated with detection buffer, for example, for about 5 minutes. NBT/BCIP dye (5-Bromo-4-Chloro-3′-Indolyphosphate p-Toluidine Salt and NBT Nitro-Blue Tetrazolium Chloride) diluted with detection buffer is added to the biochip, which is allowed to develop in the dark, for example, for about 1 hour to about 2 days under humid conditions.

The biochips are scanned, for example, using an Epson Expression 1680 Scanner (Seiko Epson Corporation, Long Beach, Calif.) at a resolution of about 1500 dpi and 16-bit grayscale. The biochip images are analyzed using Array-Pro Analyzer (Media Cybernetics, Inc., Silver Spring, Md.) software. Because the identity of the miRNA probes on the biochip are known, the sample can be identified as including particular miRNAs when spots of hybridized miRNAs-and-probes are visualized. Additionally, the density of the spots can be obtained and used to quantitate the identified miRNAs in the sample.

The identity and relative quantity of miRNAs in a sample can be used to provide an miRNA profiles for a particular sample. An miRNA profile for a sample includes information about the identities of miRNAs contained in the sample, quantitative levels of miRNAs contained in the sample, and/or changes in quantitative levels of miRNAs relative to another sample. For example, an miRNA profile for a sample includes information about the identities, quantitative levels, and/or changes in quantitative levels of miRNAs associated a particular cellular type, process, condition of interest, or other cellular state. Such information can be used, for diagnostic purposes, drug development, drug screening and/or drug efficacy testing. In an embodiment, the miRNAs of the present invention are upregulated in subjects having pre-clinical EAC and BE. For example, the presence of these miRNAs in high levels compared with controls indicates a diagnosis of BE or EAC in a subject.

In another example, with regard to diagnostics, if it is known that the presence or absence of a particular miRNA or group of miRNAs is associated with the presence or absence of a particular condition of interest, then a diagnosis of the condition can be made by obtaining the miRNA profile of a sample taken from a patient being diagnosed.

### EXAMPLES

Tissue Specimens. All patients provided written informed consent under a protocol approved by the Institutional Review Boards at the University of Maryland and Baltimore Veterans Affairs Medical Centers, where all endoscopies were performed. Biopsies were taken using a standardized biopsy protocol. Research tissues were obtained from macroscopically apparent Barrett's epithelium or from mass lesions in patients manifesting these changes at endoscopic examination, and histology was confirmed using parallel aliquots from identical locations obtained at the same endoscopy. All biopsy specimens were stored in liquid nitrogen prior to DNA/RNA extraction.

miRNA extraction from serum. TRIzol LS Reagent (Invitrogen, cat. no. 15596-018) was used to extract total RNA from sera of 16 patients with EAC, BE or 12 age-matched normal EGD, and 16 tissues each of EAC, BE or 12 age-matched normal EGD. 750 μl of TRIzol LS Reagent was added to 250 μl of serum sample and mixed thoroughly. After 5 minutes of incubation, 200 μl of chloroform is added to the mixture, followed by 3 minutes of incubation. Then, the mixture was centrifuged at 12,000×g for 15 minutes at 4° C. After centrifugation, the upper aqueous layer was transferred into new tubes, and 1.5 volumes of 100% ethanol was added to 1 volume of the aqueous layer. The mixture was then added to RNeasy Mini kit (QIAGEN, cat. no. 74904) columns for the total RNA extraction according to the manufacturer's instructions. 30 μl of RNase-free water was added onto the column to elute the RNA.

Quantitative RT-PCR (qRT-PCR) is an invaluable tool for highly sensitive and accurate quantitation of miRNA expression, and constitutes the standard method for independently validating microarray data. The application of TaqMan (I RT-PCR technology permits the analysis of mature miRNAs, rather than their precursors, ensuring the biological relevance of miRNA expression.

Gene Expression Microarrays. Arrays containing 60-mer oligonucleotide probes corresponding to 22,000 genes (Illumina HumanRef-8 Expression BeadChip v2, Illumina, San Diego, Calif.) were used to construct an mRNA expression database for the cell lines studied. 100 ng of total RNA was used for each labeling and hybridization reaction. Data was normalized according to the LOWESS fitting curve method using MATLAB (The MathWorks, Inc., Natick, Mass.).

MicroRNA Microarrays. MiRNA Labeling Reagent and Hybridization Kits (Agilent, Santa Clara, Calif.) and Agilent's Human miRNA Microarray V1 which contains 471 human miRs, were used to generate global miR expression profiles. This platform is designed to ensure extremely high data fidelity and robustness. Each miR is represented by 30 probes on the array (i.e., 15 replicates of 2 distinct probes hybridize to each miR). Furthermore, these 30 probes are evenly distributed across the array to minimize positional hybridization bias. 100 ng of total RNA from each cell line was phosphatase-treated and then labeled with cyanine 3-pCp. The labeled RNA was purified using Micro Bio-spin columns (BIO-RAD, Hercules, Calif.) and subsequently hybridized to a human miR microarray slide at 55° C. for 20 hours. After hybridization, the slides were washed with Gene Expression Wash Buffer (Agilent) and scanned on an Agilent Microarray Scanner (Agilent) using Agilent's Scan Control, version A. 7.0.1 software. Data was collected and normalized to non-functional small RNA internal controls.

Statistical Analysis. Results of experiments were displayed as mean±standard deviation. To evaluate statistical significance, Student's unpaired t test was used, unless otherwise noted.

Quantitative RT-PCR for miR Expression. TaqMan MicroRNA Assays, Human (Applied Biosystems, Foster City, Calif.) were used to confirm miR expression changes identified on miR microarrays, according to the manufacturer's protocol. qRT-PCR was performed in triplicate. RNU6B (RNU6B TaqMan microRNA Assay kit, Applied Biosystems) was used as an internal control.

### Example 1

MiR microarrays are hybridized to miRs extracted from matching tissues and blood obtained from 16 subjects each with esophageal adenocarcinoma (EAC), and compared to that of 12 healthy subjects.

In addition to these samples, miRs extracted from various normal esophageal, Barrett's, and EAC cell lines (HEEPiC, CHTRT, GiHTRT, QHTRT, and OE33 from ATCC, Manassas, Va.) were also used. For these experiments, we used QIAGEN's miRNeasy Mini Kit for the actual miR extraction, and Agilent's Human miRNA Microarray V1 which contains 471 human miRs.

### Example 2

MiR-array data generated was normalized either by Agilent's GeneSpring GX 11.5 software or by the array control small RNA called Hurs. The normalized data was analyzed using significance analysis of microarrays (SAM).

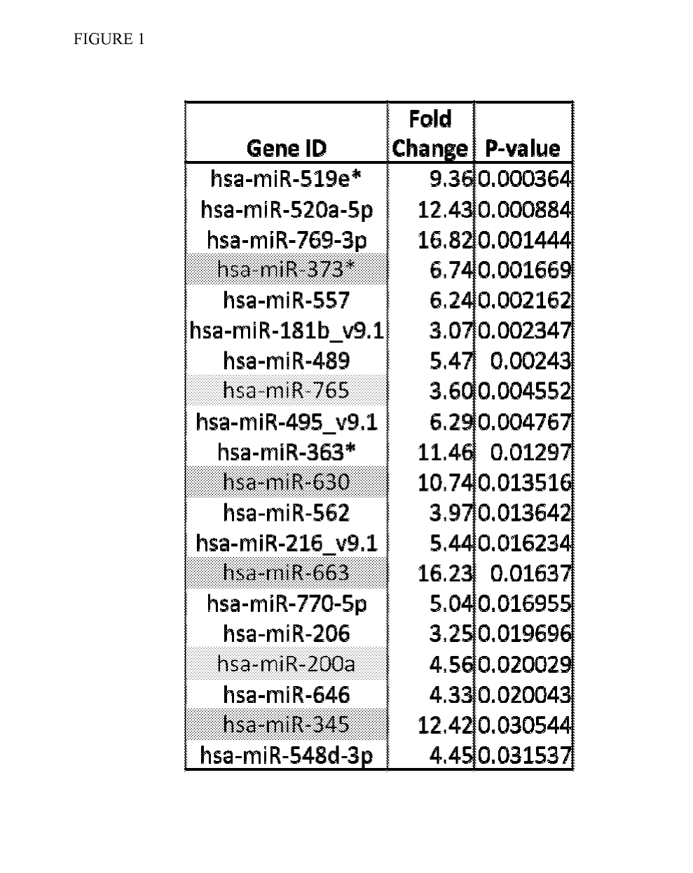

The serum data was first normalized using the Hurs array control (FIG. 1). The top 144 highest fold-change overexpressed miRs were selected that differed by a significant p-value between diseased and normal control (NC). As a final filtering criterion, to ensure that serum miRs will be robustly detectable, miRs were chosen whose individual serum levels uniformly exceeded array background by at least a factor of 5.

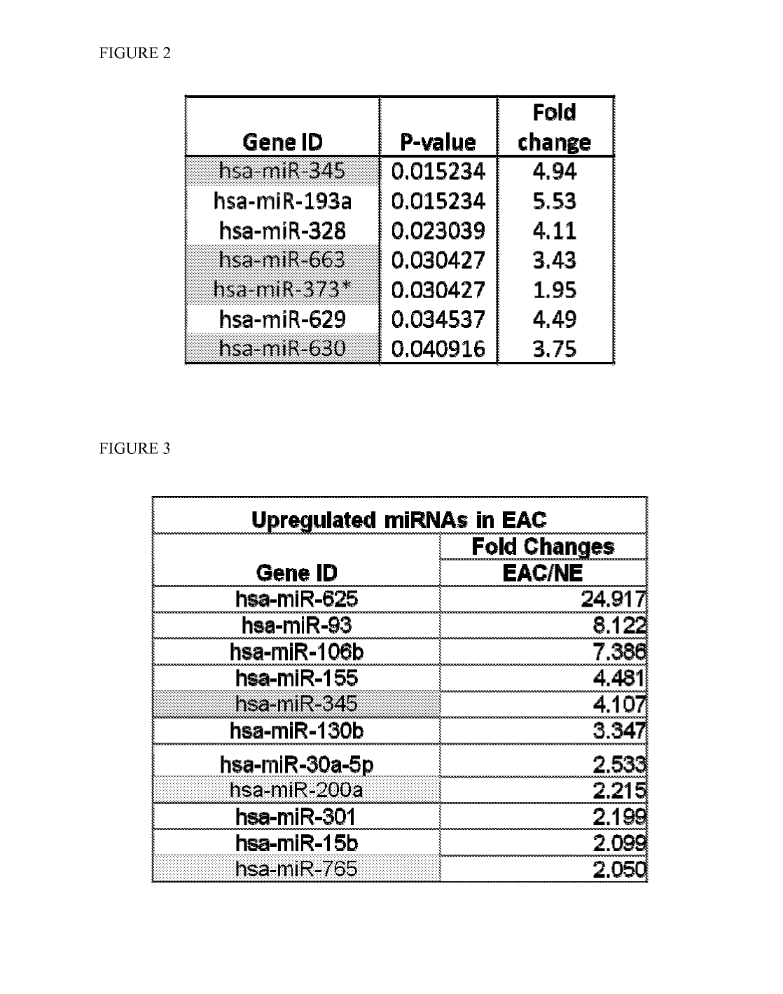

Next, the same data was normalized using GeneSpring GX 11.5 software, which used percentile shift normalization. This procedure generated an initial 7 possible miR candidates (FIG. 2).

### Example 3

The cell line data from various normal esophageal, Barrett's, and EAC cell lines (HEEPiC, CHTRT, GiHTRT, QHTRT, and OE33) was processed in the same way as the serum data in Example 2. The cell line data SAM result generated 11 possible miR candidates (FIG. 3). We arrived at a selection of 14 miR candidates (hsa-miR-200a, hsa-miR-345, hsa-miR-373*, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301a, hsa-miR-15b) which commonly appeared or significant in 3 separate analysis.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An array of oligonucleotide probes for identifying miRNAs in a sample, comprising probes that each selectively bind a mature miRNA; and a platform; wherein the probes are immobilized on the platform; wherein at least two probes selectively bind a human miRNA selected from human miRNAs comprising sequences of SEQ ID NOS: 1-7 and 10-14 or portions or fragments thereof; or at least two probes are selected from probes comprising sequences of SEQ ID NOS: 1-7 and 10-14 or portions or fragments thereof.

2. The array of claim 1, and further comprising at least one randomly-generated oligonucleotide probe sequence used as a negative control; at least one oligonucleotide sequence derived from a housekeeping gene, used as a negative control for total RNA degradation; at least one randomly-generated sequence used as a positive control; and a series of dilutions of at least one positive control sequence used as saturation controls; wherein at least one positive control sequence is positioned on the array to indicate orientation of the array.

3. The array of claim 2, wherein the human micro-RNAs are selected from the group consisting of hsa-miR-200a (SEQ ID NO: 1), hsa-miR-345 (SEQ ID NO: 2), hsa-miR-373 (SEQ ID NO: 3), hsa-miR-630 (SEQ ID NO: 4), hsa-miR-663 (SEQ ID NO: 5), hsa-miR-765 (SEQ ID NO: 6), hsa-miR-625 (SEQ ID NO: 7), hsa-miR-93 (SEQ ID NO: 8), hsa-miR-106b (SEQ ID NO: 9), hsa-miR-155 (SEQ ID NO: 10), hsa-miR-130b (SEQ ID NO: 11), hsa-miR-30a (SEQ ID NO: 12), hsa-miR-301a (SEQ ID NO: 13), hsa-miR-15b (SEQ ID NO: 14) or portions or fragments thereof of any of the miRNAs.

4. A biochip comprising a solid substrate further comprising at least two oligonucleotide probes of claim 3, which are capable of hybridizing to a target sequence under stringent hybridization conditions and attached at spatially defined address on the substrate.

5. A method of determining oncogenic, cancerous, premalignant or metaplastic changes the esophagus or gastrointestinal tract of a mammalian subject comprising:
(a) extracting miRNA from a sample obtained from a mammalian subject;
(b) contacting the miRNA from (a) with the array of claim 3;
(c) performing an analysis using the array of b) to determine expression of at least one miRNA obtained from the sample; and
(d) comparing the expression of at least two or more miRNA obtained from the sample tissue with the expression of at least one miRNA obtained from a control sample,
wherein a detectable change in the expression of at least two or more miRNA obtained from the sample compared to control is indicative of oncogenic, cancerous, premalignant or metaplastic changes in the gastrointestinal tract of a mammalian subject.

6. The method of claim 5, wherein the sample obtained from a mammalian subject is selected from the group consisting of: blood, serum and plasma.

7. A method of staging the oncogenic, cancerous, premalignant or metaplastic changes in the esophagus or gastrointestinal tract of a mammalian subject comprising:
a) obtaining a sample from the subject;
b) contacting the RNA from (a) with the array of claim 3;
c) determining the amount of at least two miRNA selected from the group consisting of hsa-miR-200a, hsa-miR-345, hsa-miR-373, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301a, hsa-miR-15b or portions or fragments thereof of any of these miRNAs, or the amount of a precursor molecule of the at least one miRNA in the sample from the subject;
d) comparing the amount of the at least two miRNA or the amount of a precursor molecule of the at least two miRNA of a) with at least one or more reference or control amounts; and
wherein when a detectable change in the amount of at least two microRNA obtained from the sample compared to the reference or control, the stage of the oncogenic, cancerous, premalignant or metaplastic changes in the gastrointestinal tract of a mammalian subject is determined.

8. The method of claim 5 wherein when the amount of two or more miRNA identified are increased over the amount of control miRNA, it is indicative of the development of esophageal adenocarcinoma (EAC) or the condition known as Barrett's esophagus (BE).

9. A method of determining oncogenic, cancerous, premalignant or metaplastic changes the esophagus or gastrointestinal tract of a mammalian subject comprising:
(a) extracting RNA from a sample obtained from a mammalian subject;
(b) determining the expression of at least two miRNA obtained from the sample; and
(c) comparing the expression of at least two miRNA obtained from the sample tissue with the expression of at least one miRNA obtained from a control sample,
wherein a detectable change in the expression of at least one miRNA obtained from the sample compared to control is indicative of oncogenic, cancerous, premalignant or metaplastic changes in the gastrointestinal tract of a mammalian subject.

10. The method of claim 9, wherein the miRNAs are selected from the group consisting of hsa-miR-200a, hsa-miR-345, hsa-miR-373, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301a, hsa-miR-15b or portions or fragments thereof, and combinations thereof.

11. The method of claim 10, wherein the oncogenic, cancerous, premalignant or metaplastic changes are indicative of the development of esophageal adenocarcinoma (EAC) or the condition known as Barrett's esophagus (BE).

12. The method of claim 11, wherein the sample obtained from a mammalian subject is selected from the group consisting of: blood, serum and plasma.

13. A method of staging the oncogenic, cancerous, premalignant or metaplastic changes in the esophagus or gastrointestinal tract of a mammalian subject comprising:
a) obtaining a sample from the subject;
b) determining the amount of at least two miRNA selected from the group consisting of hsa-miR-200a, hsa-miR-345, hsa-miR-373, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301a, hsa-miR-15b or portions or fragments thereof, or the amount of a precursor molecule of the at least two miRNA in the sample from the subject;
c) comparing the amount of the at least two miRNA or the amount of a precursor molecule of the at least two miRNA of a) with at least one or more reference or control amounts; and
wherein when a detectable change in the amount of at least two miRNA obtained from the sample compared to the reference or control, the stage of the oncogenic, cancerous, premalignant or metaplastic changes in the gastrointestinal tract of a mammalian subject is determined.

14. A method for diagnosing the progression the oncogenic, cancerous, premalignant or metaplastic changes in the esophagus or gastrointestinal tract of a mammalian subject comprising:
a) obtaining a sample from the subject;
b) determining the amount of at least two miRNA selected from the group consisting of hsa-miR-200a, hsa-miR-345, hsa-miR-373, hsa-miR-630, hsa-miR-663, hsa-miR-765, hsa-miR-625, hsa-miR-93, hsa-miR-106b, hsa-miR-155, hsa-miR-130b, hsa-miR-30a, hsa-miR-301a, hsa-miR-15b or portions or fragments thereof, or the amount of a precursor molecule of the at least two miRNA in the sample from the subject;
c) comparing the amount of the at least two miRNA or the amount of a precursor molecule of the at least two miRNA of a) with at least one or more reference or control amounts; and
wherein when a detectable change in the amount of at least two miRNA obtained from the sample compared to the reference or control, the progression of the oncogenic, cancerous, premalignant or metaplastic changes in the gastrointestinal tract of a mammalian subject is determined.

15. (canceled)
